(19) United States
(12) Patent Application Publication
(10) Pub. No.: US 2006/0013802 A1
(43) Pub. Date: Jan. 19, 2006

(54) Techniques to treat neurological disorders by enhancing the presence of anti-inflammatory mediators

(75) Inventor: Lisa L. Shafer, Stillwater, MN (US)

Correspondence Address: MEDTRONIC, INC., 710 MEDTRONIC PARK, MINNEAPOLIS, MN, 55432-9924, US

(73) Assignee: MEDTRONIC, INC., Minneapolis, MN (US)

(21) Appl. No.: 11/152,944

(22) Filed: Jun. 15, 2005

Related U.S. Application Data
(63) Continuation-in-part of application No. 10/972,177, filed on Oct. 22, 2004.
(63) Continuation-in-part of application No. 10/972,157, filed on Oct. 22, 2004.
(60) Provisional application No. 60/514,137, filed on Oct. 24, 2003.
(60) Provisional application No. 60/638,633, filed on Dec. 22, 2004.

(51) Int. Cl. A61K 38/20; G01N 33/53; A61K 9/22

(52) U.S. Cl. 424/85.2; 604/890.1; 435/7.1

(57) ABSTRACT

Methods and devices to enhance anti-inflammatory effects in the CNS to treat neurological, neurodegenerative, neuropsychiatric disorders, pain and brain injury are described. Anti-inflammatory enhancing agents that target IL-10 production and signal transduction pathways are discussed. Devices described include therapy delivery devices comprising a reservoir capable of housing an anti-inflammatory enhancing agent and a catheter operably coupled to the device and adapted to deliver the agent to a target site within a subject.

### RELATED APPLICATIONS

This application is a continuation-in-part application of application Ser. No. 10/972,177, filed Oct. 22, 2004 and Ser. No. 10/972,157, filed Oct. 22, 2004, both of which claim the benefit of priority to Provisional Application Ser. No. 60/514,137, filed Oct. 24, 2003. This application also claims the benefit of priority to Provisional Application Ser. No. 60/638,633, filed Dec. 22, 2004. Each of the above-mentioned applications is incorporated herein by reference in their respective entireties.

### FIELD

This disclosure relates to medical devices and methods for enhancing the presence or effect of anti-inflammatory mediators, particularly for treatment of neurological, neurodegenerative, neuropsychiatric disorders, pain, and brain injury.

### BACKGROUND

Inflammation and neurodegeneration that is characteristic of neurodegenerative disease, chronic pain, and traumatic brain injury may progress even when the initial cause of neuronal degeneration or insult has disappeared. It is believed that toxic substances released by the neurons or glial cells may be involved in the propagation and perpetuation of neuronal degeneration. Neuronal degeneration, pain and other disease pathology in the central nervous system (CNS) has been attributed to the toxic properties of pro-inflammatory cytokines, such as tumor necrosis factor alpha or beta (TNF), interleukin (IL)-1 beta, and interferon (IFN)-gamma. Moreover, neuronal degeneration and other disease pathology in the CNS can be attributed to a lack of sufficient quantities of anti-inflammatory cytokines, such as IL-10, IL-4, IL-9, IL-11, IL-13 and transforming growth factor beta (TGF-beta). Therapies aimed at increasing the amount of anti-inflammatory cytokines, particularly IL-10, may overcome the effects of pro-inflammatory cytokines and thereby attenuate the pathology associated with chronic pain, neurodegenerative diseases, traumatic brain injury and abnormal glial physiology. Furthermore, enhancing the constitutive levels of anti-inflammatory cytokines may provide a prophylactic therapy for individuals at risk for, or at early stages of, a certain disease or condition of the brain.

Interleukin-10 suppresses pro-inflammatory cytokine production (IL-1beta, IL-6, TNFalpha, IL-8, G-CSF, GM-CSF), T-cell proliferation and the antigen presenting capacity of specific cell types. IL-10 promotes the proliferation and differentiation of B cells important for adequate defense against foreign agents. In general, IL-10 inhibits all the activities that favor the inflammatory or specific cellular immune response and enhances those activities that are associated with adaptive immunity as well as scavenger function. IL-10 represents a substantial suppressor of the cellular immunology and reduces the production of pro-inflammatory molecules and several adhesion molecules usually resulting in the blockade, or at least dampening, of an inflammatory cascade. Frequently, the pro-inflammatory TNF-initiated cascade has deleterious effects at the cellular, tissue and organ level. The inhibitory effects of IL-10 in the periphery are widely understood. Only recently has its effects in the CNS begun to be elucidated.

As in the periphery, IL-10 is a powerful suppressor of cellular immune responses with a postulated role in brain inflammation. In vitro and some in vivo experiments have demonstrated that IL-10 exerts a concentration dependent prevention of neuronal damage induced by excitotoxicity in various neuron populations (Grilli et al 2000). Other researchers have demonstrated IL-10's neuroprotective effect in animal models by peripheral (intraperitoneal) administration of exogenous IL-10 (Mesples et al, 2003) or by using gene therapy approaches (Koeberle et al, 2004). Furthermore, in vitro and some in vivo experiments demonstrated that IL-10 may suppress the production of beta-amyloid peptides and inflammatory proteins surrounding senile plaque deposits (Szcepanik et al., 2001). Szcepanik and Ringheim (2003) later demonstrated that IL-10 administered to mice intravenously (i.v.) was moderately effective at inhibiting cytokines and chemokines that are typically elevated in and around neurotic plaque areas in Alzheimer's diseased brains. In vivo experiments in pain models suggest that gene therapy (Milligan, 2005a) or acute (single injection) intrathecal administration of recombinant IL-10 briefly reverses pain behaviors (Milligan, 2005b). However, this reversal of pain behavior was short-lived due to the short half life of IL-10. Together these results suggest that IL-10 may have neuroprotective and immunoprotective roles in the CNS. However, therapeutic effects in the CNS appear to be hampered by the route of administration and inadequate delivery system.

To date, recombinant cytokines are typically administered in the periphery and are not readily capable of penetrating the blood-brain-barrier. However, systemic delivery of an anti-inflammatory cytokine, especially in its recombinant protein form, is associated with the risk of serious side-effects, such as immuno-suppression and opportunistic infections. Furthermore, acute (or single injection) CNS delivery is likely inadequate to achieve a marked therapeutic effect (Mesples et al, 2003; Milligan et al, 2005b).

Anti-inflammatory cytokines typically exist as either a transmembrane or soluble protein. Due to the short half-life of these proteins, past administration techniques have demonstrated inadequate therapeutic success along with a risk of serious side effects. As an alternative to administering exogenous recombinant protein, it has been suggested that augmentation of endogenous IL-10 production may be achieved using modified cells, viral vectors, Epstein-Bar virus, plasmid DNA and the like. These techniques are described to increase a cells ability to synthesize IL-10 protein in vivo and thereby provide a therapy for inflammatory conditions. For example, adenoviral vectors encoding IL-10 transiently reverse pain behaviors (Milligan, 2005a; Milligan, 2005b). However, this reversal was short-lived and likely not optimal for therapeutic use. Additionally, Gene therapy approaches rely on the alteration of cellular machinery to synthesize the protein, are unlikely to be regulatable, and may have immunogenic consequences. Furthermore, it is likely that the therapeutic value and safety of an IL-10 enhancing strategy relies on the availability of native (active) protein at a specific, target location and being able to regulate its presence throughout the course of the immune response, disease or pain condition.

U.S. Pat. No. 5,368,854A1 describes traditional methods of parenteral administration for IL-10 protein to treat inflammatory bowel disease either alone or in combination with other therapeutic reagents but does not describe administration to the CNS. Others disclose methods to purify bacterially expressed IL-10 protein. WO9744057A1 describes a method for continuous delivery of cytokines, for example IL-10, via a biocompatible capsule containing encapsulated cells. The capsule is implanted in its entirety in the intrathecal space of the CNS and the IL-10 is eluted as a product of the encapsulated cells. WO9744057A1 does not describe any method of IL-10 delivery beyond cellular delivery of IL-10. WO0108717A1 discloses a drug-polymer microsphere to control release of a down-regulatory cytokine such as IL-10. The drug-polymer microspheres may steadily or intermittently release the protein. However, the polymer microsphere delivery device does not offer the precision or the ability to modify the therapeutic required. In addition, WO0108717A1 does not describe the use of such a device for therapeutic use in CNS disorder. WO0238035A2 discloses a method to treat pain by administering a compound that decreases IL-1beta activity to the CNS, which is one of the effects of IL-10. The compounds disclosed in WO0238035A2 may inhibit the activity of a MAP kinases or a transcription factor such as CREB. WO0238035A2 does not describe the use of a delivery device and instead specifies the use of a therapeutic agent that crosses the blood brain barrier when administered to the periphery. Several other mechanisms of delivering recombinant human (rh)IL-10 protein have been described for peripheral inflammatory diseases and are summarized in Pharmacological Reviews “Interleukin-10 Therapy-Review of a New Approach” vol 55, no 2, 2003. However, the administration of rhIL-10 either alone or in combination with other anti-inflammatory therapeutic agents using a drug delivery system has not been previously described. Nor has a delivery device and method of delivery been described to deliver such agents to the CNS to treat neurodegenerative disease, chronic pain, brain injury and other inflammatory conditions in the CNS.

Systemic administration of recombinant human IL-10 (ILODECAKIN, Schering-Plough) has been developed and evaluated in a variety of inflammatory diseases including Crohn's disease, rheumatoid arthritis, psoriasis, chronic hepatitis C, and acute pancreatitis. Results of these clinical trials regarding the administration of recombinant IL-10 revealed a good safety profile but only marginal efficacy in each disease application. A major problem in realizing the potential of IL-10 therapies has been adequate delivery of IL-10 to the site of action. Taking into account the adverse effects of high-dose IL-10 and its short half-life in vivo, local and regulatable delivery regimen is essential for optimal efficacy. The poor efficacy in previous attempts of IL-10 therapy approaches can be primarily attributed to two insufficiencies. First, the failures are likely due to inadequate dosing regimen. Second, IL-10 is known to act locally, making the effect of IL-10 highly dependent upon the tissue micro-environment. As a result, systemic administration appears to be inadequate.

None of the publications mentioned above teaches an adequate delivery system for the administration of any anti-inflammatory protein agents, or prescribes brain sites for effective administration of anti-inflammatory protein agents. In addition, they do not suggest how the dosage of anti-inflammatory protein agents can be effectively regulated during infusion. Significantly, the data concerning the administration of IL-10 in a variety of diseases that contain an inflammatory process has been conflicting in some instances. It is possible that the different therapeutic outcomes are dependent on the mode of delivery, the local microenvironment, the disease stage and the IL-10 concentration.

### BRIEF SUMMARY

This disclosure describes the administration of anti-inflammatory cytokines and describes methods and devices to enhance IL-10 and other anti-inflammatory mediators in the CNS to treat neurological, neurodegenerative, neuropsychiatric disorders, pain and brain injury. Potentially safer and more efficacious means of administration, as well as potentially safer and more efficacious agents aimed at enhancing IL-10, and more generally, enhancing the net effect of an anti-inflammatory response are discussed. These and the manner of administration are considered as second generation therapies to the previously proposed gene therapy anti-inflammatory approaches for use in pain. However, the approach of delivering anti-inflammatory cytokines in their protein form, using a programmable implantable delivery system has not been previously described for use in the brain or spinal cord or to treat CNS disorders.

An embodiment of the invention provides a system for treating a CNS disorder associated with inflammation or an inflammatory agent in a subject in need thereof. The system comprises a device having a reservoir adapted to house a therapeutic composition, a catheter coupled to the device and adapted for administering the therapeutic composition to the CNS of the subject, and a CNS disorder treating amount of a therapeutic composition. The system may include a pump operably coupled to the reservoir and the catheter to cause the therapeutic composition to flow from the reservoir through the catheter. The pump may be implantable and may be a programmable pump, a fixed rate pump, a variable rate pump, and the like. The system may also include a sensor. The sensor may be coupled to a device to adjust one or more infusion parameters, for example flow rate and chronicity. The sensor may be capable of detecting a dysfunctional immune or pain response, or whether an inflammatory condition has been attenuated or enhanced, and the like. The therapeutic composition comprises an anti-inflammatory mediator, such as IL-10, in an amount effective to treat the CNS disorder. The therapeutic agent may be administered directly to the CNS (intrathecally, intracerebroventricularly, intraparenchymally, etc.). Delivery of the therapeutic compositions may be regulated by use of one or more of a programmable pump, closed-loop feedback control, a selectable rate valve, and the like.

Embodiments of the invention provide systems and methods for the administration of a therapeutic composition comprising a combination of anti-inflammatory mediators that enhance the level of IL-10. In an embodiment, a system for administration of a therapeutic composition comprising one or more anti-inflammatory mediator that, alone or together, enhances the level of IL-10 is a “controlled administration system”. A “controlled administration system” is a direct and local administration system to deliver the combination of agents in a controlled manner. A controlled administration system may be a pump system, such as a peristaltic pump, an osmotic pump, a piston pump, and the like. An infusion pump may be implantable and may be a programmable pump, a fixed rate pump, a variable rate pump, and the like. A catheter is operably connected to the pump and configured to deliver the combination of agents to a target tissue region of a subject.

In an embodiment, the invention provides a method for treating a CNS disorder associated with an inflammatory response, in a subject in need thereof. The method comprises administering to the subject an anti-inflammatory agent, designed to reduce an adverse immune response by enhancing the presence of an anti-inflammatory agent in an amount effective to treat the CNS disorder. The anti-inflammatory mediator may be administered directly to the subject's CNS. The method may further comprise administering an anti-inflammatory mediator to enhance the treatment of the CNS disorder.

In an embodiment of the invention, recombinant human IL-10 protein may be delivered in combination with one or more additional agent that is capable of modulating other aspects of the inflammatory response. Non-limiting examples of suitable additional agents include IL-4, IL-9, IL-11, IL-13, and TGF-β whose predominate functions are anti-inflammatory in nature but in some cases may be pro-inflammatory

Various embodiments of the invention may provide one or more advantages. For example, as discussed herein, enhancing the concentration of IL-10 by means of administering a protein has several advantages over genomic approaches aimed at enhancing the cell's (either transplanted or endogenous) ability to synthesize anti-inflammatory cytokines. The goal of enhancing the presence of IL-10 through the addition of its protein form may provide greater efficacy, greater specificity of effect, greater regulation, and avoid potentially deleterious effects of genomic insertions. Furthermore, more than one anti-inflammatory agent may be used in combination in order to raise the net effect of total available anti-inflammatory agents directed at inhibiting various pathways incurred in an inflammatory response or CNS disease condition. These and other advantages will become evident to those of skill in the art upon reading the description provided herein.

### BRIEF DESCRIPTION OF THE DRAWINGS

FIG. 1 is a schematic diagram of the IL-10 signal transduction pathway.

FIG. 2 is a diagrammatic illustration of a drug delivery system according to an embodiment of the present invention.

FIG. 3 is a diagrammatic illustration of a patient's brain, the associated spaces containing cerebrospinal fluid, and the flow of cerebrospinal fluid in the subarachnoid space.

FIG. 4 is a diagrammatic illustration of a drug delivery system and a catheter implanted in a patient according to an embodiment of the present invention.

FIG. 5 is a diagrammatic illustration of a catheter implanted in a patient and a drug delivery system according to an embodiment of the present invention.

FIG. 6 is a diagrammatic illustration of a drug delivery system and catheter implanted in a patient according to an embodiment of the present invention.

FIG. 7 is a diagrammatic illustration of a drug delivery system comprising a sensor according to an embodiment of the present invention.

The figures are not necessarily to scale.

### DETAILED DESCRIPTION OF THE INVENTION

In the following descriptions, reference is made to the accompanying drawings that form a part hereof, and in which are shown by way of illustration several specific embodiments of the invention. It is to be understood that other embodiments of the present invention are contemplated and may be made without departing from the scope or spirit of the present invention. The following detailed description, therefore, is not to be taken in a limiting sense.

All scientific and technical terms used in this application have meanings commonly used in the art unless otherwise specified. The definitions provided herein are to facilitate understanding of certain terms used frequently herein and are not meant to limit the scope of the present disclosure.

In the context of the present invention, the terms “treat”, “therapy”, and the like mean alleviating, slowing the progression, preventing, attenuating, or curing the treated disease.

As used herein, “disease”, “disorder”, “condition” and the like, as they relate to a subject's health, are used interchangeably and have meanings ascribed to each and all of such terms.

As used herein, “subject” means a mammal undergoing treatment. Mammals include mice, rats, cats, guinea pigs, hamsters, dogs, horses, cows, monkeys, chimpanzees, and humans.

As used herein, “anti-inflammatory enhancing agent” means an agent that is capable of enhancing the effects of IL-10, IL-4 or other endogenous anti-inflammatory molecules, such as IL-9, IL-11, IL-13, Transforming Growth Factor beta (TGF-β), and the like, and includes intracellular anti-inflammatory modifying agents and extracellular anti-inflammatory agents, as well as IL-10 signal transduction modulating agents and IL-10 inducing agents.

As used herein, “extracellular anti-inflammatory enhancing agent” means an agent that affects the action of anti-inflammatory cytokines, such as IL-10, IL-4, IL-9, IL-11, IL-13, Transforming Growth Factor beta (TGF-β), and the like, at a anti-inflammatory cytokine cell surface receptor and agents that affect the action of secreted molecules associated with the anti-inflammatory cascade such as antibodies and fragments thereof. Extracellular anti-inflammatory enhancing agents include small molecule chemical agents and biological agents, such as polynucleotides and polypeptides. Extracellular anti-inflammatory enhancing agents may include IL-10 signal transduction modulating agents and IL-10 enhancing agents. Non-limiting examples of extracellular anti-inflammatory enhancing agents include recombinant protein forms of IL-10, IL-4, IL-9, IL-11, IL-13, Transforming Growth Factor beta.

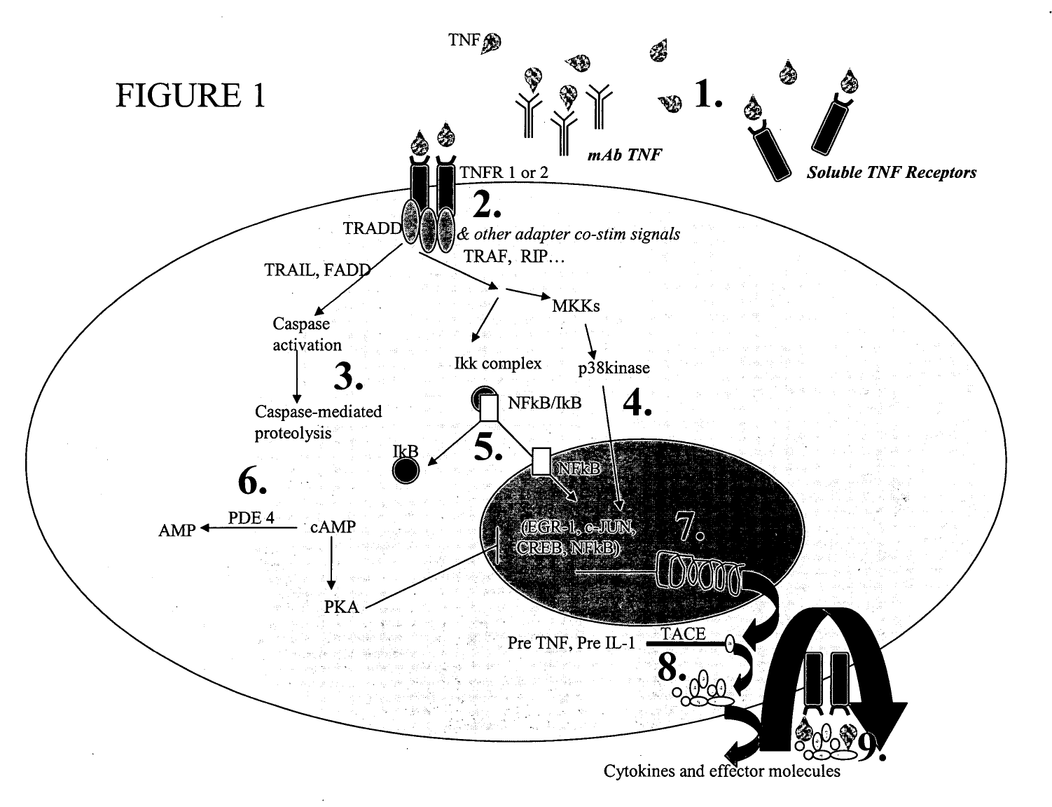

As used herein, “IL-10-signal transduction-modulating agent” means an agent that affects a molecule associated with signal transduction in the IL-10 signal transduction pathway depicted in FIG. 1. IL-10 signal transduction-modulating agents include IL-10 inducing agents. IL-10 signal transduction-modulating agents include small molecule chemical agents and biological agents, such as polynucleotides and polypeptides, which include antibodies and fragments thereof, antisense nucleotides, small interfering RNA (siRNA), and ribozymes.

As used herein, “IL-10 inducing agent” means an agent that, when administered to a subject, results in an increase in IL-10, or an active portion thereof, within the subject. Accordingly, IL-10 itself is an IL-10 inducing agent.

Delivery System

An embodiment of the invention provides a system for delivering a therapeutic composition comprising an anti-inflammatory enhancing agent to a CNS of a subject in need thereof. The system comprises therapy delivery device and a catheter operably coupled to the therapy delivery device. The therapy delivery device may be a pump device. Non-limiting examples of pump devices include fixed-rate pumps, selectable rate pumps, variable rate pumps, and the like. Any pumping mechanism may be used. For example, the pump may be any of an osmotic pump, a piston pump, a peristaltic pump, and the like. The pump device may be programmable. Each of the aforementioned pump systems comprises a reservoir for housing a fluid composition comprising an IL-10 inducing agent or IL-10 signal transduction-modulating agent. The catheter comprises one or more delivery regions, through which the fluid may be delivered to one or more target regions of the subject. The pump device may be implantable or may be placed external to the subject.

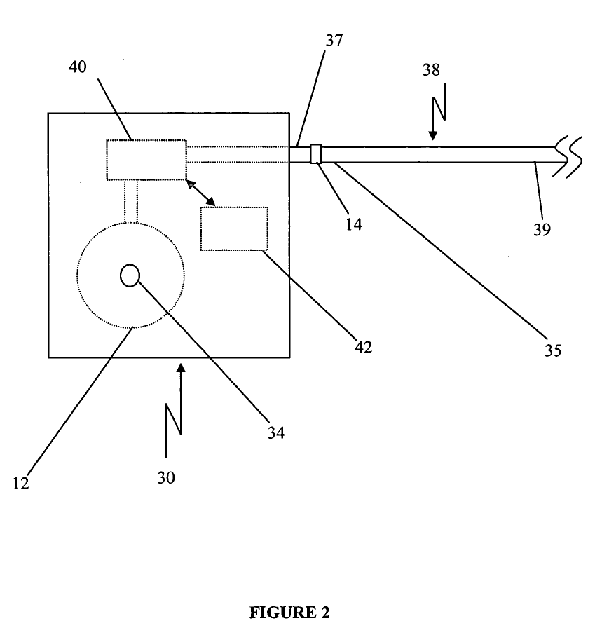

The therapy delivery device 30 shown in FIG. 2 comprises a reservoir 12 for housing a composition comprising an IL-10 inducing agent or IL-10 signal transduction-modulating agent and a pump 40 operably coupled to the reservoir 12. The catheter 38 shown in FIG. 2 has a proximal end 35 coupled to the therapy delivery device 30 and a distal end 39 adapted to be implanted in a subject. Between the proximal end 35 and distal end 39 or at the distal end 39, the catheter 38 comprises one or more delivery regions (not shown) through which the anti-inflammatory agent may be delivered. The therapy delivery device 30 may have a port 34 into which a hypodermic needle can be inserted to inject a quantity of anti-inflammatory agent into reservoir 12. The therapy delivery device 30 may have a catheter port 37, to which the proximal end 35 of catheter 38 may be coupled. The catheter port 37 may be operably coupled to reservoir 12. A connector 14 may be used to couple the catheter 38 to the catheter port 37 of the therapy delivery device 30. The therapy delivery device 30 may be operated to discharge a predetermined dosage of the pumped fluid into a target region of a patient. The therapy delivery device 30 may contain a microprocessor 42 or similar device that can be programmed to control the amount of fluid delivery. The programming may be accomplished with an external programmer/control unit via telemetry. A controlled amount of fluid comprising an anti-inflammatory enhancing agent may be delivered over a specified time period. With the use of a programmable delivery device 30, different dosage regimens may be programmed for a particular patient. Additionally, different therapeutic dosages can be programmed for different combinations of fluid comprising therapeutics. Those skilled in the art will recognize that a programmed therapy delivery device 30 allows for starting conservatively with lower doses and adjusting to a more aggressive dosing scheme, if warranted, based on safety and efficacy factors.

If it is desirable to administer more than one therapeutic agent the fluid composition within the reservoir 12 may contain a second, third, fourth, etc. therapeutic agent. Alternatively, the device 30 may have more than one reservoir 12 for housing additional compositions comprising a therapeutic agent. When the device 30 has more than one reservoir 12, the pump 40 may draw fluid from one or more reservoirs 12 and deliver the drawn fluid to the catheter 38. The device 30 may contain a valve operably coupled to the pump 40 for selecting from which reservoir(s) 12 to draw fluid. Further, one or more catheters 38 may be coupled to the device 30. Each catheter 38 may be adapted for delivering a therapeutic agent from one or more reservoirs 12 of the pump 40. A catheter 38 may have more than one lumen. Each lumen may be adapted to deliver a therapeutic agent from one or more reservoirs 12 of the device 30. It will also be understood that more than one device 30 may be used if it is desirable to deliver more than one therapeutic agent. Such therapy delivery devices, catheters, and systems include those described in, for example, copending application Ser. No. 10/245,963, entitled IMPLANTABLE DRUG DELIVERY SYSTEMS AND METHODS, filed on Dec. 23, 2003, which application is hereby incorporated herein by reference.

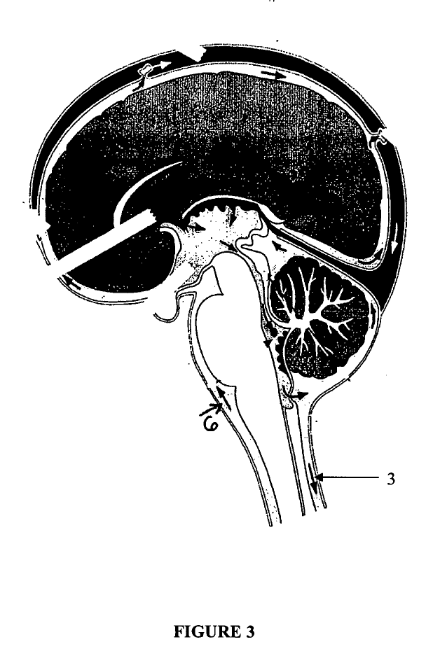

According to an embodiment of the invention, a composition comprising an anti-inflammatory enhancing agent may be delivered directly to cerebrospinal fluid 6 of a subject. Referring to FIG. 3, cerebrospinal fluid (CSF) 6 exits the foramen of Magendie and Luschka to flow around the brainstem and cerebellum. The arrows within the subarachnoid space 3 in FIG. 3 indicate cerebrospinal fluid 6 flow. The subarachnoid space 3 is a compartment within the central nervous system that contains cerebrospinal fluid 6. The cerebrospinal fluid 6 is produced in the ventricular system of the brain and communicates freely with the subarachnoid space 3 via the foramen of Magendie and Luschka. A composition comprising an anti-inflammatory enhancing agent may be delivered to cerebrospinal fluid 6 of a patient anywhere that the cerebrospinal fluid 6 is accessible. For example, the composition may be administered intrathecally or intracerebroventricularly.

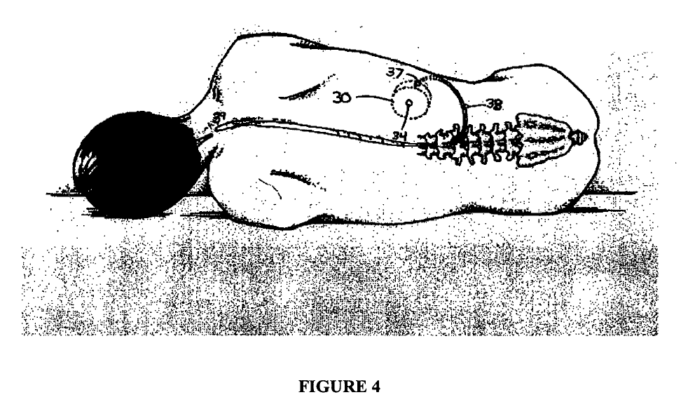

FIG. 4 illustrates a system adapted for intrathecal delivery of a composition comprising an anti-inflammatory enhancing agent. As shown in FIG. 4, a system or device 30 may be implanted below the skin of a patient. Preferably the device 30 is implanted in a location where the implantation interferes as little as practicable with patient activity. One suitable location for implanting the device 30 is subcutaneously in the lower abdomen. According to an embodiment of the invention, catheter 38 may be positioned so that the distal end 39 of catheter 38 is located in the subarachnoid space 3 of the spinal cord such that a delivery region (not shown) of catheter is also located within the subarachnoid space 3. It will be understood that the delivery region can be placed in a multitude of locations to direct delivery of a therapeutic agent to a multitude of locations within the cerebrospinal fluid 6 of the patient. The location of the distal end 39 and delivery region(s) of the catheter 38 may be adjusted to improve therapeutic efficacy. While device 30 is shown in FIG. 4, delivery of a composition comprising an anti-inflammatory enhancing agent into the CSF can be accomplished by injecting the therapeutic agent via port 34 to catheter 38.

According to an embodiment of the invention, a composition comprising an anti-inflammatory enhancing agent may be delivered intraparenchymally directly to brain tissue of a subject. A therapy delivery device may be used to deliver the agent to the brain tissue. A catheter may be operably coupled to the therapy delivery device and a delivery region of the catheter may be placed in or near a target region of the brain.

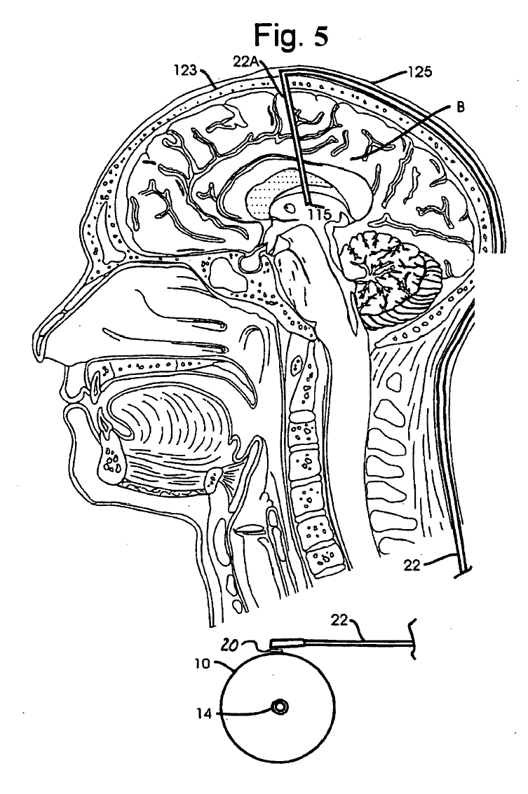

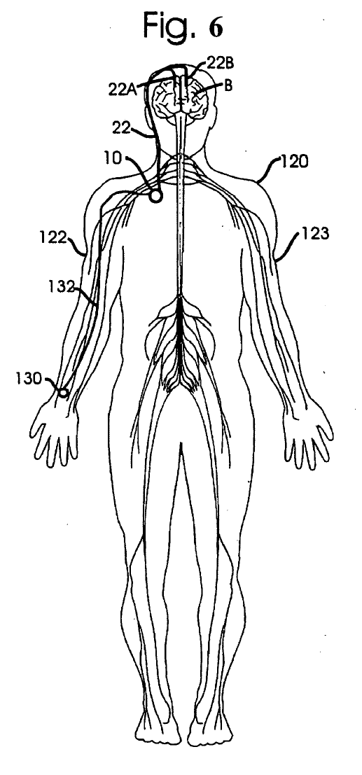

One suitable system for administering a therapeutic agent to the brain is discussed in U.S. Pat. No. 5,711,316 (Elsberry) as shown FIGS. 5 and 6 herein. Referring to FIG. 5, a system or therapy delivery device 10 may be implanted below the skin of a patient. The device 10 may have a port 14 into which a hypodermic needle can be inserted through the skin to inject a quantity of a composition comprising a therapeutic agent. The composition is delivered from device 10 through a catheter port 20 into a catheter 22. Catheter 22 is positioned to deliver the agent to specific infusion sites in a brain (B). Device 10 may take the form of the like-numbered device shown in U.S. Pat. No. 4,692,147 (Duggan), assigned to Medtronic, Inc., Minneapolis, Minn. The distal end of catheter 22 terminates in a cylindrical hollow tube 22A having a distal end 115 implanted into a target portion of the brain by conventional stereotactic surgical techniques. Additional details about end 115 may be obtained from pending U.S. application Ser. No. 08/430,960 entitled “Intraparenchymal Infusion Catheter System,” filed Apr. 28, 1995 in the name of Dennis Elsberry et at. and assigned to the same assignee as the present application. Tube 22A is surgically implanted through a hole in the skull 123 and catheter 22 is implanted between the skull and the scalp 125 as shown in FIG. 1. Catheter 22 is joined to implanted device 10 in the manner shown, and may be secured to the device 10 by, for example, screwing catheter 22 onto catheter port 20.

Referring to FIG. 6, a therapy delivery device 10 is implanted in a human body 120 in the location shown or may be implanted in any other suitable location. Body 120 includes arms 122 and 123. Catheter 22 may be divided into twin tubes 22A and 22B that are implanted into the brain bilaterally. Alternatively, tube 22B may be supplied with drugs from a separate catheter and pump.

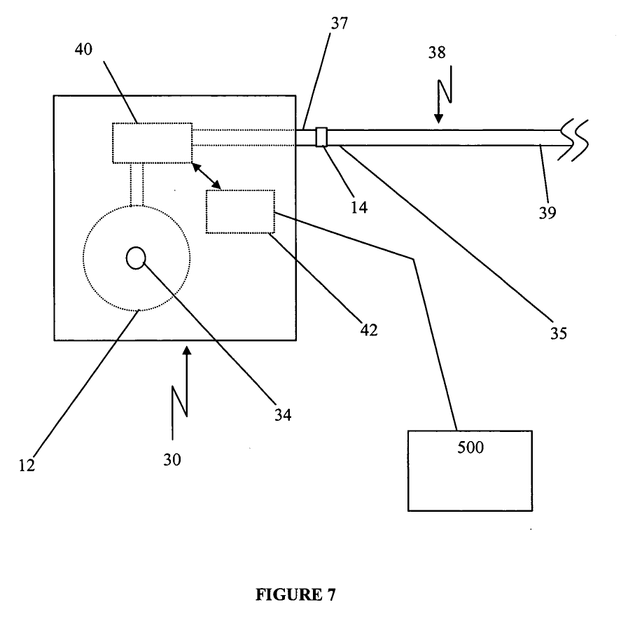

Referring to FIG. 7, therapy delivery device 30 may include a sensor 500. Sensor 500 may detect an event associated with a CNS disorder associated with an inflammatory immune response, such as a dysfunctional immune or sickness response, or treatment of the disorder, such as or whether an immune response has been attenuated or enhanced. Sensor 500 may relay information regarding the detected event, in the form of a sensor signal, to processor 42 of device 30. Sensor 500 may be operably coupled to processor 42 in any manner. For example, sensor 500 may be connected to processor via a direct electrical connection, such as through a wire or cable. Sensed information, whether processed or not, may be recoded by device 30 and stored in memory (not shown). The stored sensed memory may be relayed to an external programmer, where a physician may modify one or more parameter associated with the therapy based on the relayed information. Alternatively, based on the sensed information, processor 42 may adjust one or more parameters associated with therapy delivery. For example, processor 42 may adjust the amount and timing of the infusion of an anti-inflammatory agent. Any sensor 500 capable of detecting an event associated with the disease to be treated or an inflammatory immune response may be used. Preferably, the sensor 500 is implantable. It will be understood that two or more sensors 500 may be employed.

Sensor 500 may detect a polypeptide associated with a CNS disorder or an inflammatory immune response; a physiological effect, such as a change in membrane potential; a clinical response, such as blood pressure; and the like. Any suitable sensor 500 may be used. In an embodiment, a biosensor is used to detect the presence of a polypeptide or other molecule in a patient. Any known or future developed biosensor may be used. The biosensor may have, e.g., an enzyme, an antibody, a receptor, or the like operably coupled to, e.g., a suitable physical transducer capable of converting the biological signal into an electrical signal. In some situations, receptors or enzymes that reversibly bind the molecule being detected may be preferred. In an embodiment, sensor 500 is capable of detecting a cytokine, such as the level of IL-10 or TNF in cerebrospinal fluid. In an embodiment, sensor 500 may be a sensor as described in, e.g., U.S. Pat. No. 5,978,702, entitled TECHNIQUES OF TREATING EPILEPSY BY BRAIN STIMULATION AND DRUG INFUSION, which patent is hereby incorporated herein by reference in its entirety, or U.S. patent application Ser. No. 10/826,925, entitled COLLECTING SLEEP QUALITY INFORMATION VIA A MEDICAL DEVICE, filed Apr. 15, 2004, which patent application is hereby incorporated herein by reference in its entirety, or U.S. patent application Ser. No. 10/820,677, entitled DEVICE AND METHOD FOR ATTENUATING AN IMMUNE RESPONSE, filed Apr. 8, 2004.

In an embodiment, cerebrospinal levels of a cytokine are detected. A sample of CSF may be obtained and the levels of, e.g., TNF or IL-10 in the sample may be detected by Enzyme-Linked Immunoabsorbant Assay (ELISA), microchip, conjugated fluorescence or the like. Feedback to a therapy delivery device may be provided to alter infusion parameters of the therapeutic agents.

In another embodiment, cerebrospinal levels of a biomarker that is diagnostic for a pain condition are detected. A sample of CSF may be obtained and the levels of neurotransmitters and neuropeptides e.g., glutamate, CCK, galanin, neuropeptide Y in the sample may be detected by Enzyme-Linked Immunoabsorbant Assay (ELISA), microchip, conjugated fluorescence or the like. Feedback to a therapy delivery device may be provided to alter infusion parameters of the therapeutic agents.

Anti-Inflammatory Enhancing Agents

Any anti-inflammatory enhancing agent capable of treating a CNS disorder, alone or in combination with one or more additional therapeutic agents, may be delivered to a subject in need thereof according to the teachings of this disclosure. The anti-inflammatory enhancing agent may be any agent that is capable of enhancing the effects of an anti-inflammatory molecule, such as IL-10, IL-4 or other endogenous anti-inflammatory molecules, including the anti-inflammatory molecules themselves or derivatives or active fragments thereof.

In an embodiment, the anti-inflammatory enhancing agent is an intracellular anti-inflammatory modifying agent. The intracellular anti-inflammatory modifying agent may be any agent that induces the sequence of intracellular events associated with a cascade associated with an anti-inflammatory cytokine, such as IL-10, IL-4, other endogenous anti-inflammatory molecules, and the like. In an embodiment, the anti-inflammatory enhancing agent is an extracellular anti-inflammatory enhancing agent. The extracellular anti-inflammatory enhancing agent may be any agent that affects the action of anti-inflammatory cytokines at a anti-inflammatory cytokine cell surface receptor or may be any agent that affects the action of secreted molecules associated with the anti-inflammatory cascade.

Other potential inducers of IL-10 production are 3-thia fatty acids, Peroxisome Proliferator Activated Receptor (PPAR-α) agonists, cannabinoid receptor agonists, thymadine dinucleotides, imidocarb, glatiramir acetate, and annexin-1. Many such IL-10 inducing agents have been developed or are currently in development for peripheral administration to treat peripheral diseases and conditions that are manifested by an abnormal immune response. However, the administration of these types of agents to targeted areas in the brain or spinal cord has not been suggested previously as a way to treat or prevent conditions associated with brain injury, pain, neurological, neuropsychiatric, and neurodegenerative disease.

IL-10 and IL-10 receptors are expressed in the brain by various cell types including astrocytes, neurons, monocytes, microglia and blood vessels. Biologic or small molecule drug therapeutic agents designed to enhance the IL-10 cascade in these cell populations may have a therapeutic or prophylactic effect in diseases and conditions of the central nervous system. The short serum half-life of recombinant human IL-10 (2-3 hrs) suggests that a delivery system capable of chronic and programmable administration may be advantageous for use in CNS disorders. Whereas others have attempted to modify the rhIL-10 formulation by conjugating the recombinant protein to various stabilizing agents, the current invention describes targeted administration approach using a pump and reservoir that protect the protein from degradation. As a result significantly lower doses may be administered when applying targeted administration. Doses could be in the range of 0.1 ng/kg to 10 μg/kg. In fact, results from rhIL-10 administration to patient's with Crohn's disease and rheumatoid arthritis, suggest that higher concentrations were less effective, due to the stimulation of TNFα production in T cells or modulation of Fc receptor expression. Such immunopotentiating effects may have contributed to the lack of therapeutic efficacy of rhIL-10 in the past. This evidence suggests that the administration of rhIL-10 would greatly benefit from a programmable pump with targeted delivery capability.

In an embodiment, IL-10 is co-administered with one or more additional therapeutic agent. When delivered via a pump system, the additional therapeutic agent may be placed in the same reservoir as IL-10 or different reservoir. The additional therapeutic agent may be delivered within the therapeutic window of the IL-10, immediately before, or immediately after, sequential or simultaneous. Examples of such additional therapeutic agents include corticosteroids, sulphasalazine, derivatives of sulphasalazine, immunosuppresive drugs such as cyclosporin A, mercaptopurine, and azathioprine, soluble TNF inhibitors (i.e. etanercept, infliximab, adimulab, CDP 870), related cytoplasmic proteins (i.e. RDP58), anti-apoptotic agents (i.e. Activase, Retavase, Pexelizumab, CAB2, RSR13), kinase inhibitors (i.e. Gleevec, Herceptin, Iressa, imatinib, herbimycin A, tyrphostin 47, and erbstatin, genistein, staurosporine), NfκB inhibitors, phosphodiesterase inhibitors, or siRNA of TNF.

Injectable Composition

The above-mentioned anti-inflammatory enhancing agent may be administered to a subject's CNS as injectable compositions. Injectable compositions include solutions, suspensions, dispersions, and the like. Injectable solutions or suspensions may be formulated according to techniques well-known in the art (see, for example, Remington's Pharmaceutical Sciences, Chapter 43, 14th Ed., Mack Publishing Co., Easton, Pa.), using suitable dispersing or wetting and suspending agents, such as sterile oils, including synthetic mono- or diglycerides, and fatty acids, including oleic acid.

Solutions or suspensions comprising a therapeutic agent may be prepared in water, saline, isotonic saline, phosphate-buffered saline, and the like and may optionally mixed with a nontoxic surfactant. Dispersions may also be prepared in glycerol, liquid polyethylene, glycols, DNA, vegetable oils, triacetin, and the like and mixtures thereof. Under ordinary conditions of storage and use, these preparations may contain a preservative to prevent the growth of microorganisms. Pharmaceutical dosage forms suitable for injection or infusion include sterile, aqueous solutions or dispersions or sterile powders comprising an active ingredient which powders are adapted for the extemporaneous preparation of sterile injectable or infusible solutions or dispersions. Preferably, the ultimate dosage form is sterile, fluid and stable under the conditions of manufacture and storage. A liquid carrier or vehicle of the solution, suspension or dispersion may be a solvent or liquid dispersion medium comprising, for example, water, ethanol, a polyol such as glycerol, propylene glycol, or liquid polyethylene glycols and the like, vegetable oils, nontoxic glyceryl esters, and suitable mixtures thereof. Proper fluidity of solutions, suspensions or dispersions may be maintained, for example, by the formation of liposomes, by the maintenance of the required particle size, in the case of dispersion, or by the use of nontoxic surfactants. The prevention of the action of microorganisms can be accomplished by various antibacterial and antifungal agents, for example, parabens, chlorobutanol, phenol, sorbic acid, thimerosal, and the like. In many cases, it will be desirable to include isotonic agents, for example, sugars, buffers, or sodium chloride. Prolonged absorption of the injectable compositions can be brought about by the inclusion in the composition of agents delaying absorption—for example, aluminum monosterate hydrogels and gelatin. Excipients that increase solubility, such as cyclodextran, may be added.

Sterile injectable solutions may be prepared by incorporating a therapeutic agent in the required amount in the appropriate solvent with various other ingredients as enumerated above and, as required, followed by sterilization. Any means for sterilization may be used. For example, the solution may be autoclaved or filter sterilized. In the case of sterile powders for the preparation of sterile injectable solutions, the preferred methods of preparation are vacuum drying and freeze-drying techniques, which yield a powder of the active ingredient plus any additional desired ingredient present in a previously sterile-filtered solution.

Dosage

Effective dosages for use in methods as described herein can be determined by those of skill in the art, particularly when effective systemic dosages are known for a particular therapeutic agent. Dosages may typically be decreased by at least 90% of the usual systemic dose if the therapeutic agent is provided in a targeted fashion. In other embodiments, the dosage is at least 75%, at least 80% or at least 85% of the usual system dose for a given condition and patient population. Dosage is usually calculated to deliver a minimum amount of one or more therapeutic agent per day, although daily administration is not required. If more than one pharmaceutical compound or composition is administered, the interaction between the same is considered and the dosages calculated. Intrathecal dosage, for example, can comprise approximately ten percent of the standard oral dosage. Alternatively, an intrathecal dosage is in the range of about 10% to about 25% of the standard oral dosage.

CNS Disorder

Embodiments of the invention provide methods and devices for treating a CNS disorder associated with inflammation or inflammatory agent by administering to a subject a CNS disorder treating effective amount of a composition comprising an anti-inflammatory enhancing agent. CNS disorders associated with inflammation or an inflammatory agent include neurological, neurodegenerative, neuropsychiatric disorders, pain and brain injury. The anti-inflammatory enhancing agent may be administered directly to the CNS of the subject by, e.g., intrathecal (IT) delivery, intracerberalventricular (ICV) delivery, or intraparenchymal (IPA) delivery. Targeted delivery to the CNS avoids the potential for systemic immuno-suppression and other risk factors associated with systemic exposure to anti-inflammatory blocking agents. In various embodiments, the anti-inflammatory enhancing agent is delivered to the CNS using a programmable pump, which allows for controlling the rate and time at which the agent is delivered and provides the ability to stop the delivery of the agent as desired. In various embodiments, an anti-inflammatory enhancing agent is also delivered in combination with an agent that inhibits the action of a pro-inflammatory cytokine. Examples of agents that inhibit the action of a pro-inflammatory cytokines are discussed in application Ser. Nos. 10/972,177 and 10/972,157.

Examples of various CNS disorders that may be treated and preferred delivery locations of therapeutic agents for treating the disorders are provided below.

1. Stroke

Blood-brain barrier breakdown and inflammation is observed in brain following stroke. Inflammatory processes are at least partly responsible for this breakdown. Anti-inflammatory enhancing agents may be administered ICV, either chronically or transiently, following a stroke. In an embodiment, anti-inflammatory enhancing agent is administered at the location of an infarct due to stroke. The location of the infarct may be identified by MRI or other known or future developed techniques. In an embodiment, the therapeutic agent is delivered to the middle cerebral artery at an infarct location or other cerebral artery distribution. Such delivery can be accomplished by placing a delivery region of a catheter in the artery and delivering the agent through the delivery region.

In addition to the ICV delivery of an anti-inflammatory enhancing agent at or near an infarct, an anti-inflammatory enhancing agent may be delivered IPA to an area surrounding the infarct to attenuate inflammation occurring in the ischemic periphery or penumbra that may lead to neurodegeneration if left untreated.

To attenuate the degeneration that occurs in a patient with hemiperesis following stroke an anti-inflammatory enhancing agent may be placed in the posterior limb of the internal capsule, for example.

In addition, an anti-inflammatory enhancing agent may be delivered to other brain regions that may be affected due to the secondary ischemic events following stroke, including but not limited to the pons, midbrain, medulla and the like.

Additional locations where an anti-inflammatory enhancing agent may be administered to treat stroke include locations where inflammatory events secondary to the initial stroke may occur. For example middle cerebral artery stroke can produce a characteristic, cell-type specific injury in the striatum. Transient forebrain ischemia can lead to delayed death of the CA1 neurons in the hippocampus. Therefore, an anti-inflammatory enhancing agent may be delivered to the striatum or hippocampus following a stroke event.

2. Alzheimer's Disease

Brain microvessels from Alzheimer's disease (AD) patients have been shown to express high levels of pro-inflammatory cytokines. It is suggested that inflammatory processes in the brain vasculature may contribute to plaque formation, neuronal cell death and neurodegeneration associated with AD. Interelukin-10 is a known suppressor of IL-1beta, TNF, and other inflammatory cytokines. Accordingly, targeted delivery of a IL-10 inducing agent to a patient suffering from AD is contemplated herein.

In an embodiment, an anti-inflammatory enhancing agent is delivered in the vicinity of an amyloid plaque, where the inflammatory response in AD is mainly located. An anti-inflammatory enhancing agent may be administered IPA at the site of amyloid beta peptide accumulations, amyloid beta plaques, neurofibrillary tangles or other pathological sites associated with AD. For example, the affected area may be cortical or cerebellar and the plaques may be observed by imaging techniques known in the field.

Other IPA sites include the basal forebrain cholinergic system, a region that is vulnerable to degeneration in AD, the structures of the temporal lobe region, a region that is responsible for cognitive decline in AD patients, specifically the hippocampus, entorhinal cortex, and dentate gyrus.

3. Epilepsy

Blood-brain barrier breakdown and inflammation is observed in brain following seizures. Inflammatory processes are at least partly responsible for this breakdown. In addition, inflammatory cytokine production is up-regulated during seizure-induced neuronal injury. In an embodiment, an anti-inflammatory enhancing agent is administered ICV, either chronically or transiently, following a seizure episode. In an embodiment, an anti-inflammatory enhancing agent is administered IPA to a seizure focus. In an embodiment, an anti-inflammatory enhancing agent is administered IPA to an area of the brain that undergoes neuronal injury, away from a specific seizure focus. For example, in patients with intractable temporal lobe epilepsy, the CA1 region of the hippocampus undergoes pathophysiological changes associated with inflammatory processes and may ultimately result in neuronal cell loss in that region. Therefore, anti-inflammatory enhancing agents may be administered to the hippocampus in a epileptic patient. Other sites of IPA delivery are associated with brain regions affected by mesial temporal sclerosis such as the hippocampus or amygdala where evidence of inflammatory processes are often detected. Other structures in the CNS known to play a key role in the epileptogenic network such as the thalamus and subthalamic nucleus may also be targeted.

4. Depression

An anti-inflammatory enhancing agent may be administered ICV to target brain regions associated with inflammation in patients with depression. One suitable ICV location is the floor of the fourth ventricle, dorsal to the abducens nuclei, that contains serotonergic neurons.

In an embodiment, an anti-inflammatory enhancing agent is administered IPA to brain regions associated with the hypothalamic-pituitary-adrenal (HPA)-axis, as dysfunction of the HPA-axis is common in patients with depression. Furthermore, the cellular immune status in the brain regions associated with the HPA-axis is abnormal and is believed to be partly responsible for depressive symptoms. Elevations in pro-inflammatory cytokines such as TNF often found in depressed patients likely affect the normal functioning of the HPA axis. Examples of brain regions associated with the HPA-axis include, but are not limited to, the hypothalamus and the anterior pituitary gland.

In an embodiment, an anti-inflammatory enhancing agent is delivered to a brain region associated with serotonin production and output, since pro-inflammatory cytokines such as TNF may lower the circulating levels of serotonin—the mood stabilizing neurotransmitter. An anti-inflammatory enhancing agent delivered in a controlled fashion to the site of serotonin production may serve to regulate the levels of TNF and thereby modulate the levels of serotonin production in patients with depression. The main site of serotonin production in the brain is the dorsal raphe nucleus. Other clusters or groups of cells that produce serotonin located along the midline of the brainstem may be targeted with IPA delivery of an anti-inflammatory enhancing agent. Main serotonergic nuclei may be targeted including the ventral surface of the pyramidal tract, the nucleus raphe obscurans, the raphe at the level of the hypoglossal nucleus, at the level of the facial nerve nucleus surrounding the pyramidal tract, the pontine raphe nucleus, above and between the longitudinal fasiculi at the central substantia grisea, the medial raphe nucleus, or the medial lemniscus nucleus.

5. Pain

An anti-inflammatory enhancing agent may be administered to a subject to treat pain in the subject. The anti-inflammatory enhancing agent may be administered intrathecally. In an embodiment, the anti-inflammatory enhancing agent is administered perispinally, which includes epidural, anatomic area adjacent the spine, intradiscal, subcutaneous, intramuscular, and intratendon administration. Generally, an agent administered perispinally to treat pain should be administered in close enough anatomic proximity to the pain fibers associated with the pain to reach the spine or subarachnoid space surrounding the pain fibers in the spinal cord in therapeutic concentration when administered perispinally. The anti-inflammatory enhancing agent may be administered perispinally via a delivery region of a catheter. The catheter may be operably coupled to a therapy delivery device.

All patents and publications referred to herein are hereby incorporated by reference in their entirety.

What is claimed is:

1. An implantable medical device comprising:
a pump;
a reservoir operably coupled to the pump;
an anti-inflammatory enhancing agent housed in the reservoir and being deliverable to a target site in a patient in an amount effective to treat a CNS disorder; and
a catheter operably coupled to the pump and configured to deliver the intracellular anti-inflammatory agent to the target site.

2. The medical device of claim 1, wherein the pump is selected from the group consisting of a fixed rate pump, a selectable rate pump, and a variable rate pump.

3. The medical device of claim 1, wherein the pump is selected from an osmotic pump, a piston pump, and a peristaltic pump.

4. The medical device of claim 1, wherein the pump is programmable.

5. The medical device of claim 1, wherein the anti-inflammatory enhancing agent is an intracellular anti-inflammatory modifying agent or an extracellular anti-inflammatory enhancing agent

6. The medical device of claim 1, wherein the anti-inflammatory enhancing agent is an IL-10 signal transduction modifying agent.

7. The medical device of claim 6, wherein IL-10 signal transduction modifying agent is an IL-10 enhancing agent.

8. The medical device of claim 7, wherein the IL-10 enhancing agent is selected from the group consisting of IL-10; recombinant human IL-10; a cyclic AMP elevating agent; IL-9; a 3-thia fatty acids; a peroxisome proliferator activated receptor (PPAR-α) agonist; a cannabinoid receptor agonist; a thymadine dinucleotide; imidocarb; glatiramir acetate; and annexin-1.

9. The medical device of claim 8, wherein the IL-10 enhancing agent is recombinant human IL-10.

10. The medical device of claim 1, further comprising a sensor capable of detecting an event associated with the disorder or treatment of the disorder.

11. The medical device of claim 10, wherein the sensor is operably coupled to the pump.

12. The medical device of claim 11, wherein a parameter of the pump is capable of being modified by data from the sensor.

13. The medical device of claim 10, further comprising a memory operably coupled to the sensor and capable of storing sensed data.

14. The medical device of claim 10, wherein the sensor is capable of detecting a dysfunctional immune or sickness response or whether an immune response has been attenuated or enhanced.

15. A method for treating a CNS disorder associated with an inflammation or an inflammatory agent in a subject in need thereof, the method comprising:
implanting a distal portion of a catheter in a patient within the patient's central nervous system (CNS); and
delivering an anti-inflammatory enhancing agent through the catheter to the subject's CNS in an amount effective to treat the CNS disorder.

16. The method of claim 15, wherein delivering the agent to the subject's CNS comprises administering the agent intraparenchymally or intracerebroventricularly.

17. The method of claim 15, wherein the CNS disorder is a neurological disorder, a neurodegenerative disorder, a neuropsychiatric disorder, pain or brain injury.

18. The method of claim 15, wherein the CNS disorder is selected from the group consisting of stroke, Alzheimer's disease, epilepsy, and depression.

19. The method of claim 15, wherein delivering the anti-inflammatory enhancing agent through the catheter comprises pumping the agent through the catheter.

20. The method of claim 15, wherein delivering the agent to the subject's CNS comprises delivering an intracellular anti-inflammatory modifying agent or an extracellular anti-inflammatory enhancing agent to the subject's CNS.

21. The method of claim 15, wherein delivering the agent to the subject's CNS comprises delivering an IL-10 signal transduction modifying agent to the subject's CNS.

22. The method of claim 21, wherein I delivering an IL-10 signal transduction modifying agent to the subject's CNS comprises delivering an IL-10 enhancing agent to the subject's CNS.

23. The method of claim 22, wherein delivering an IL-10 enhancing agent comprises delivering an agent selected from the group consisting of IL-10; recombinant human IL-10; a cyclic AMP elevating agent; IL-9; a 3-thia fatty acids; a peroxisome proliferator activated receptor (PPAR-α) agonist; a cannabinoid receptor agonist; a thymadine dinucleotide; imidocarb; glatiramir acetate; and annexin-1.

24. The method of claim 22, wherein delivering an IL-10 enhancing agent comprises delivering recombinant human IL-10.

25. A method for treating a CNS disorder associated with a pro-inflammatory mediator in a subject in need thereof, the method comprising:
implanting a distal portion of a catheter in a patient in proximity to the patient's central nervous system (CNS); and
delivering an anti-inflammatory enhancing agent through the catheter in proximity to the subject's CNS in an amount effective to treat the CNS disorder.

26. The method of claim 25, wherein the CNS disorder is pain and delivering an anti-inflammatory enhancing agent comprises delivering the agent perispinally.

27. The method of claim 25, wherein delivering the anti-inflammatory enhancing agent through the catheter comprises pumping the agent through the catheter.
